(19) United States
(12) Patent Application Publication
(10) Pub. No.: US 2011/0318409 A1
(43) Pub. Date: Dec. 29, 2011

(54) FRACTIONS OF WHEAT GERM FERMENT

(76) Inventors: Máté Hidvégi, Budapest (HU); Gyula Bencze, Budapest (HU); György Kéri, Budapest (HU); László Õrfi, Budapest (HU)

(21) Appl. No.: 13/254,652

(22) PCT Filed: Mar. 5, 2010
(86) PCT No.: PCT/HU2010/000026

§ 371 Date: Sep. 2, 2011

(30) Foreign Application Priority Data
Mar. 6, 2009 (HU) P0900141
Sep. 9, 2009 (HU) P0900563

(51) Int. Cl. A61K 36/899; A61P 37/04; A61P 35/00; A61P 37/02; A61K 9/127; A61K 9/28

(52) U.S. Cl. 424/450; 424/115; 424/123; 424/124; 424/474; 424/278.1

(57) ABSTRACT

The invention concerns wheat germ ferment, its biologically active fractions, the process for their production, the pharmaceutical preparations containing them and their uses.

The subject of the invention concerns biologically active fractions obtained from wheat germ ferment, process for their production, immunostimulatory, immunomodulatory and antitumor pharmaceutical preparations containing them, and therapeutical procedures done with these fractions.

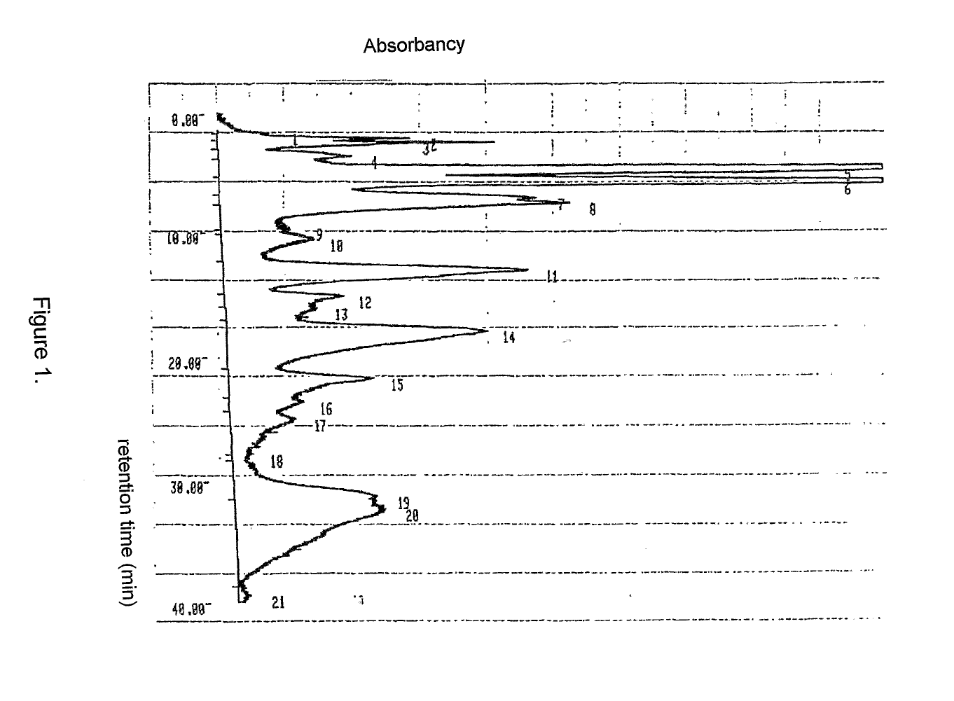

It is known that the dried material (active ingredient) obtained by fermenting wheat germ in aqueous medium in the presence of Saccharomyces cerevisiae, and by drying the fermentation liquid, has immunostimulatory and metastasis inhibiting (Hungarian Patent No. 223 344) and anti-arthritis (EP 1530645 A1) effects. The dried material is not a single compound, but a material mixture, which can approximately be characterized by the chromatogram of FIG. 1 (Hungarian Patent No. 223 344).

It is marketed under the name Avemar®. Human clinical studies proved that Avemar® is effective in the treatment of e.g. skin melanoma (Cancer Biother. Radiopharm. 2008 August; 23(4): 477-82), rheumatoid arthritis (Clin. Exp. Rheumatol. 2006 June; 24(3): 325-8), colorectal cancer (Br. J. Cancer 2003 Aug. 4; 89(3): 465-9).

Drawbacks of the use of the Avemar® product are due to the following factors: it contains 15-20% (w/w) maltodextrine—because the thermoplastic material obtained by concentrating the fermentation liquid is dried by spray-drying —, it also contains 40-45% sweetener—because of organoleptic reasons—thus, the weight of a single dose of the product is large. Also, the pharmaceutical form has to be chosen to protect the active ingredient from the moisture content of the air. It has unpleasant smell and taste which hinder its widespread use.

Recently, there is a great interest toward the regulatory function of 5′ adenosine monophosphate-activated protein kinase (AMPK) molecules in the development of various diseases. AMPK influences, among others, the energy economy of cells. For instance, it has been shown that AMPK has central regulatory role in the development and maintenance of tumor cells-specific aerobic glycolysis (Warburg effect) (Nat. Rev. Cancer. 2009 August; 9(8). 563-75). AMPK also has an important role in the development of the so called metabolic syndrome and in the associated diseases (diabetes, hypertension, obesity, arteriosclerosis, etc.) (Trends Pharmacol. Sci. 2005 February; 26(2): 69-76) (J. Physiol. 2006 Jul. 1; 574(Pt 1): 63-71).

We set the target to isolate such fractions from wheat germ ferment, which preserve the efficacy of the original dried material, or are more efficient but, are not hygroscopic and have no unpleasant smell.

Wheat germ was fermented in aqueous medium in the presence of Saccharomyces cerevisiae, and the fermentation liquid was concentrated and/or dried. For the purpose of the fermentation of wheat germ, other microorganisms belonging to the Saccharomyces genus can also be used, such as Saccharomyces bayanus and Saccharomyces boulardii, but other microorganisms used in the fermentation of foods can also be applied. Dehydration can be carried out by vacuum-drying, spray-drying, or lyophilization, preferably, lyophilization is used. The lyophilizate was tested in 18 cell lines, and those were chosen (A431 and SW480), in which cells responded satisfactorily to the concentration of 1000 mg/ml.

For separation, aqueous solutions of different pH values, and various solvents were tested, like hexane, benzene, chloroform, diethyl ether, ethyl acetate, dichloromethane, alcohols (methanol and ethanol), however, considerable dissolution was only observable with alcohols.

The material obtained after dissolution was filtered and washed by alcohol. The above procedure with the filtrate was repeated till the alcoholic phase became colorless. The alcoholic phases were united and evaporated (fraction A2).

The weight of fraction A2 was 35-40% of the starting dry material.

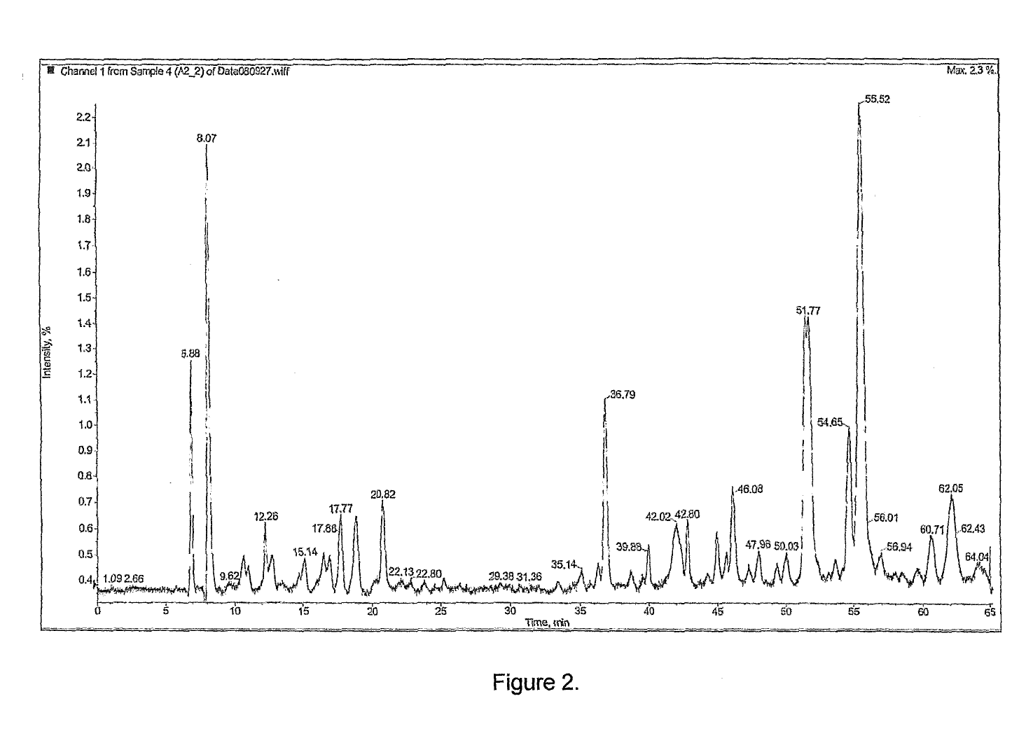

The UV chromatogram of fraction A2 is shown in FIG. 2.

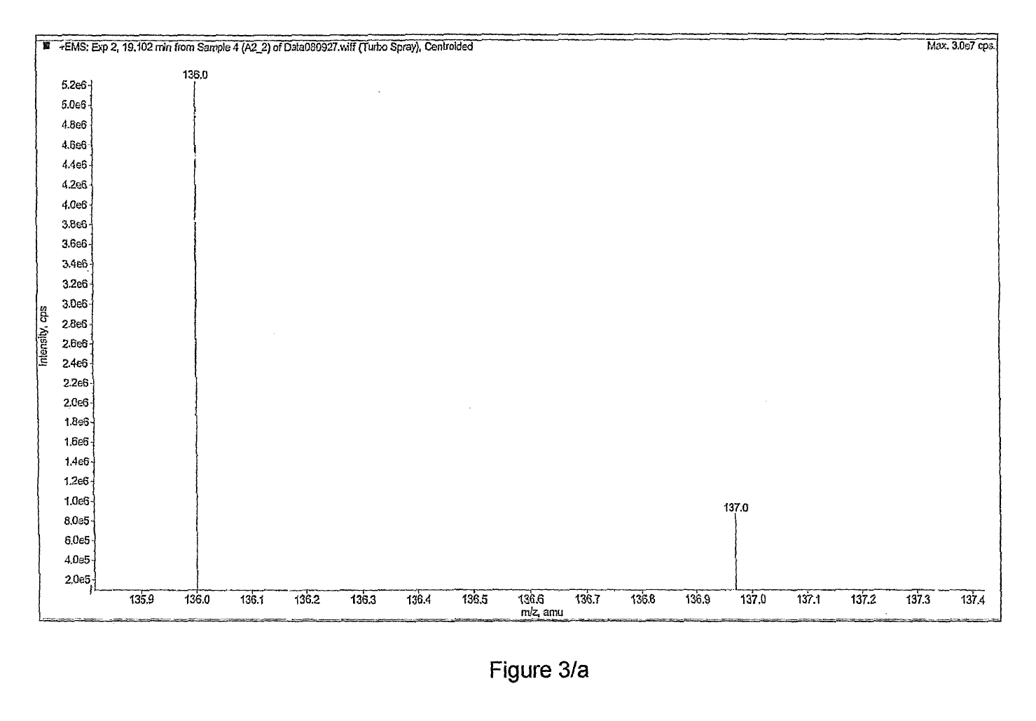

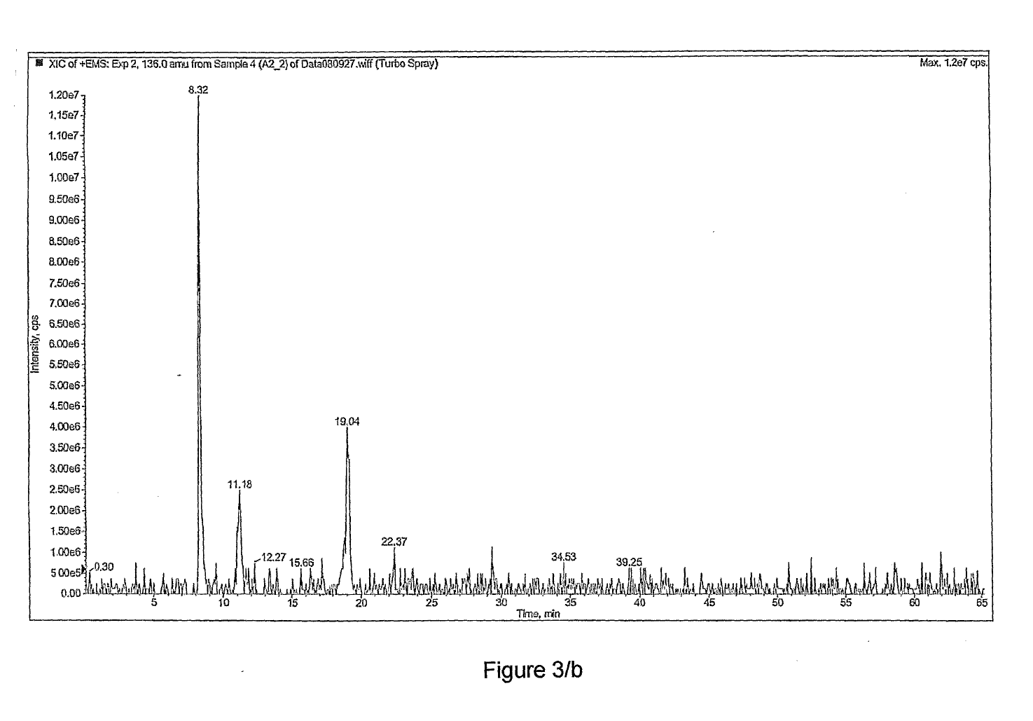

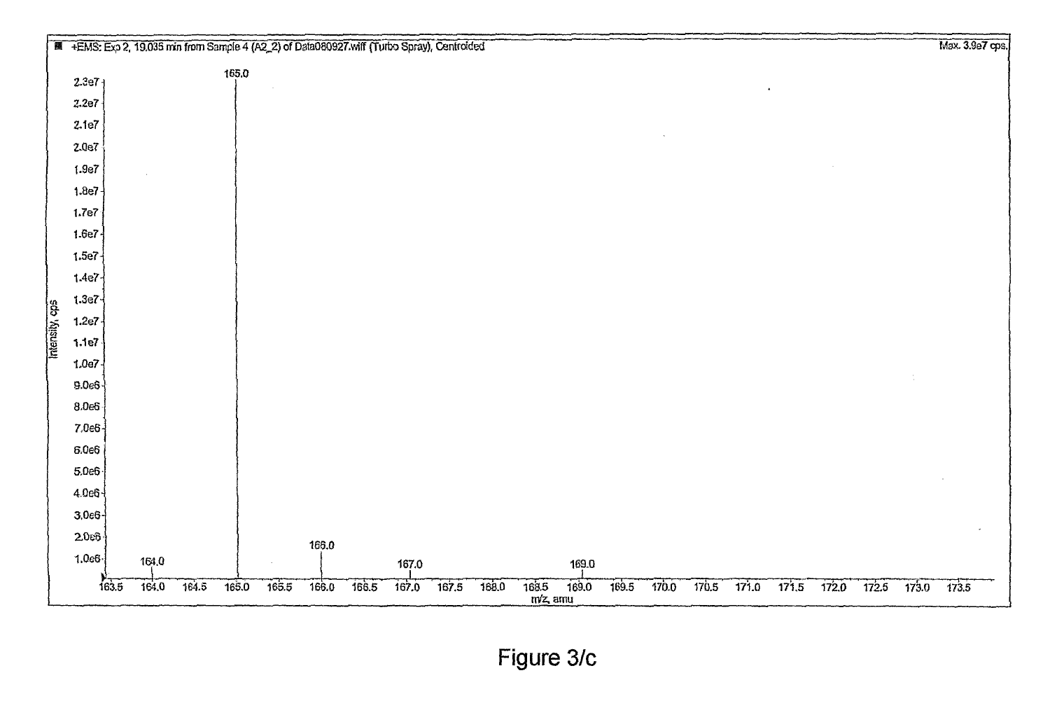

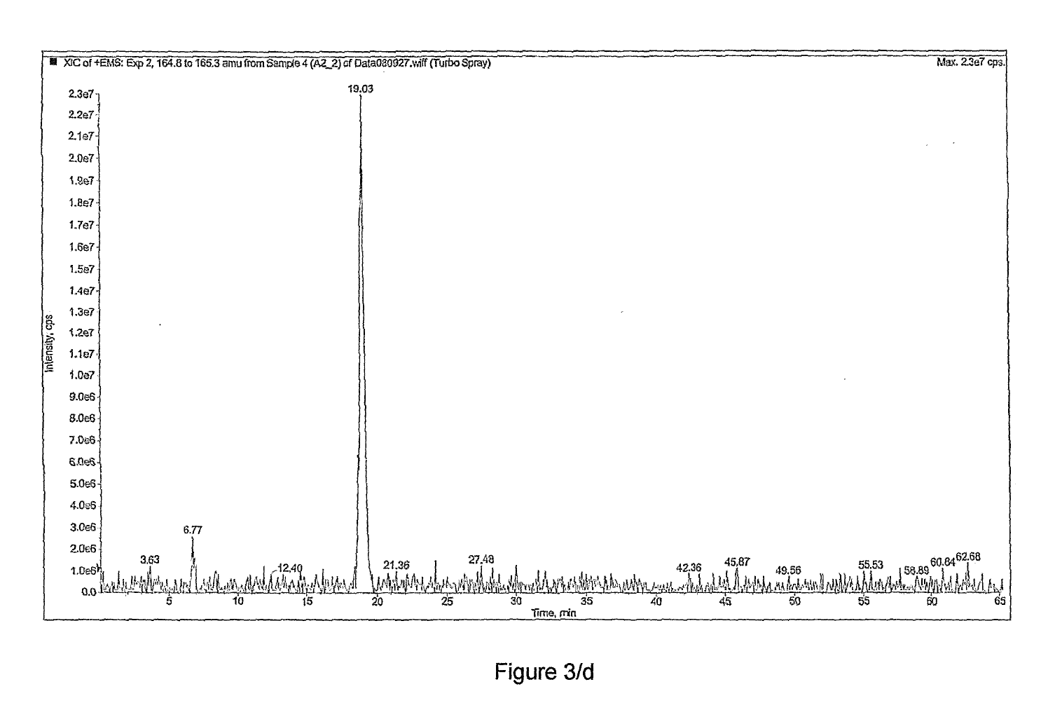

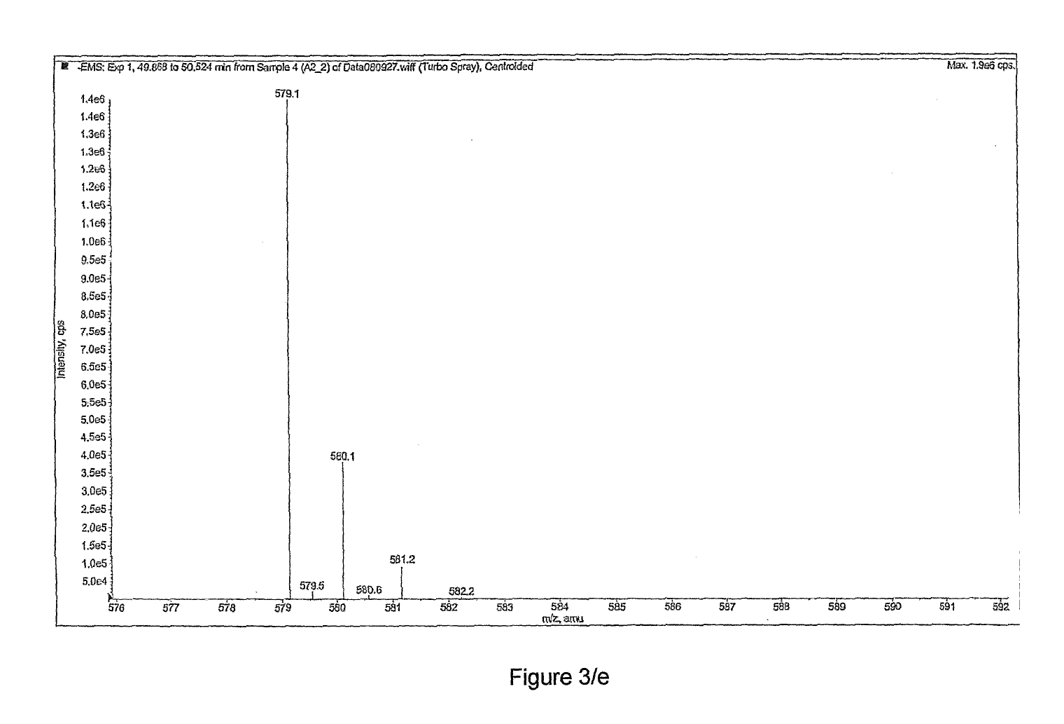

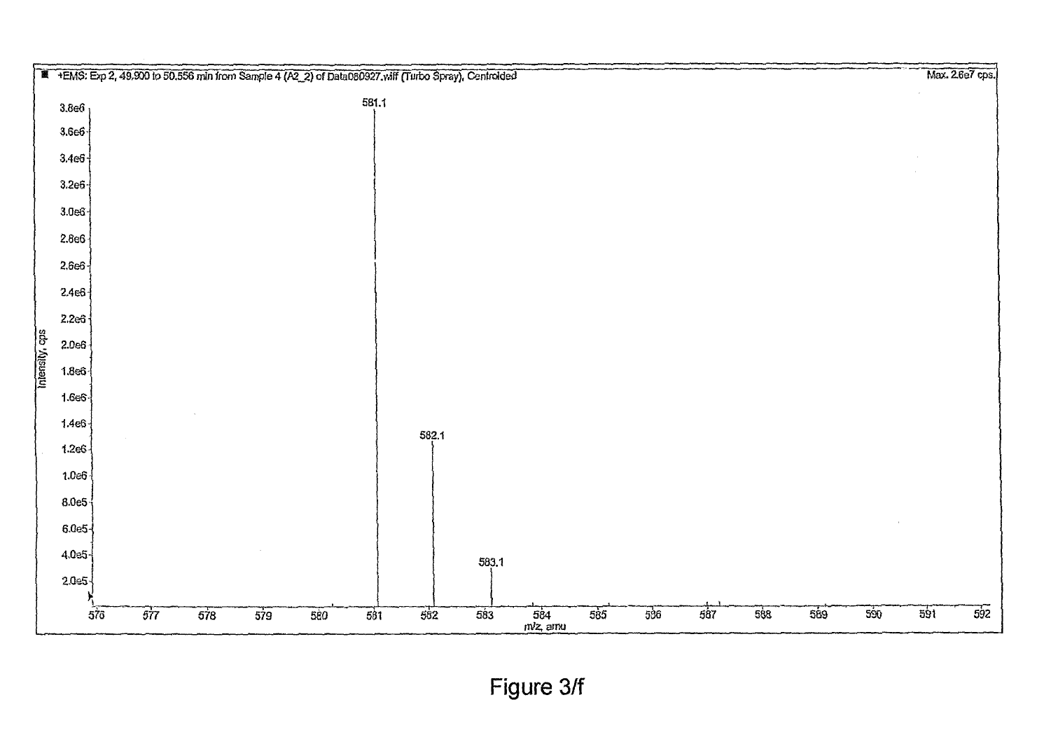

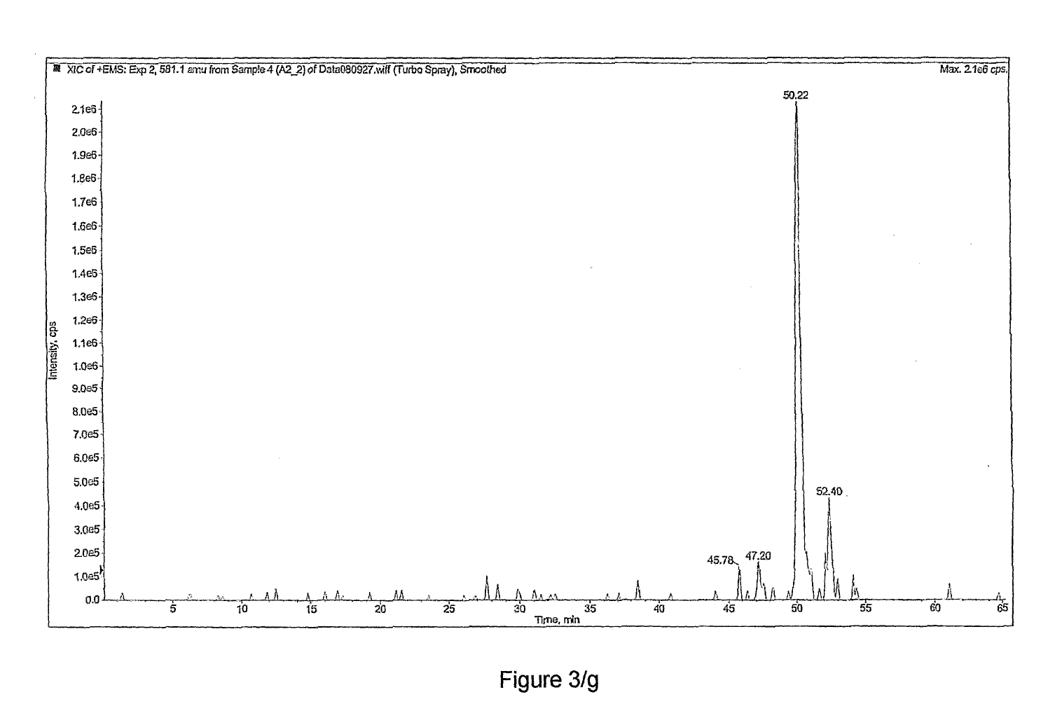

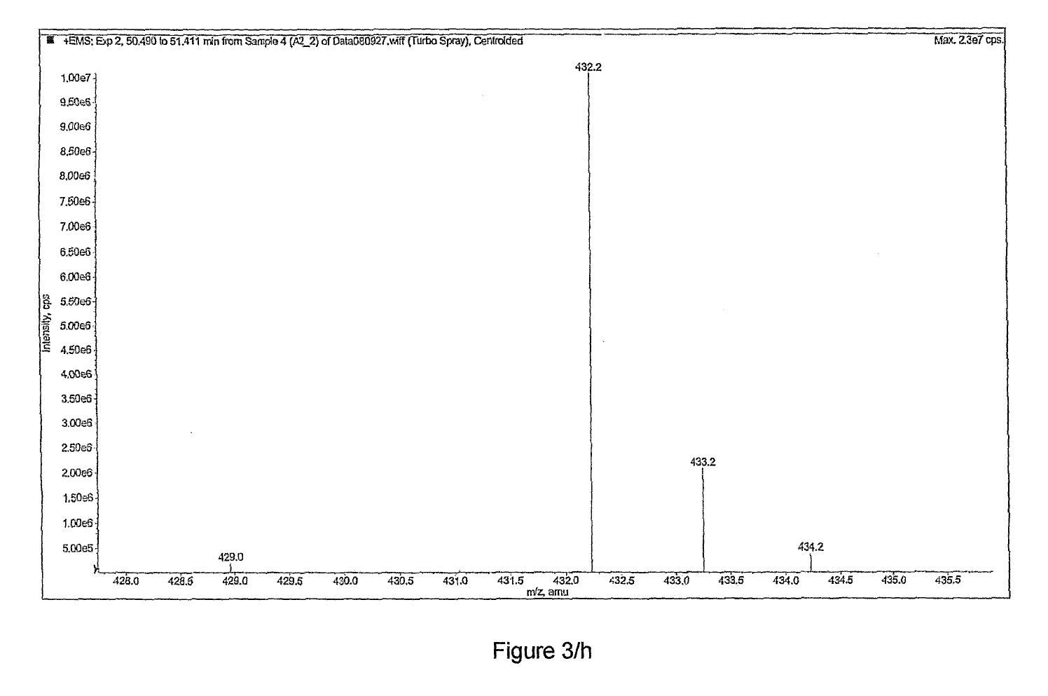

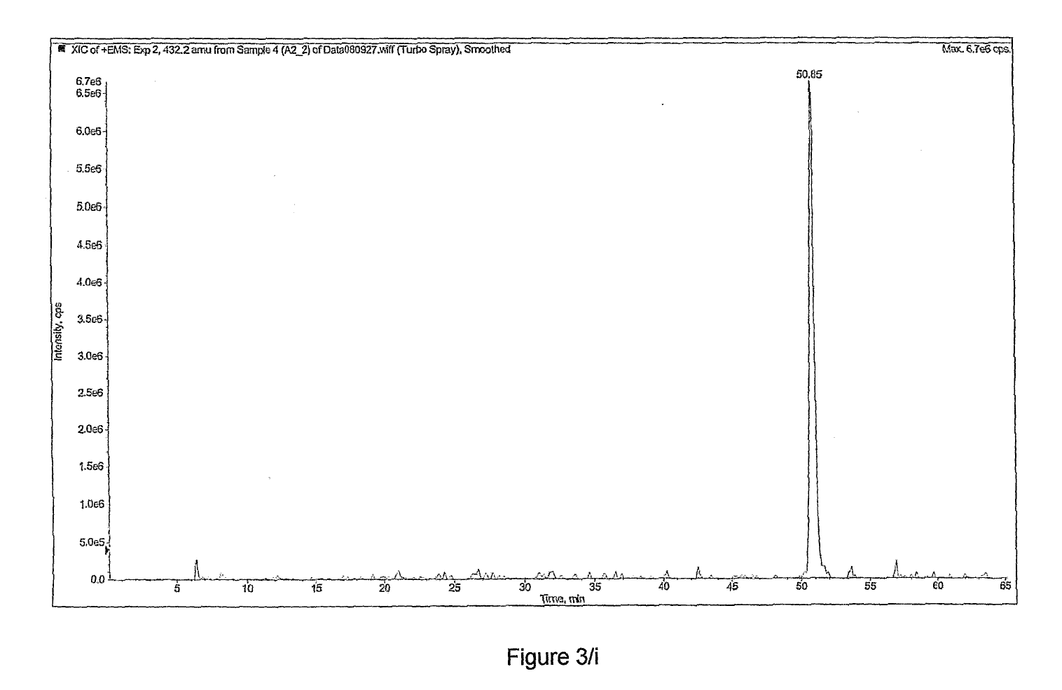

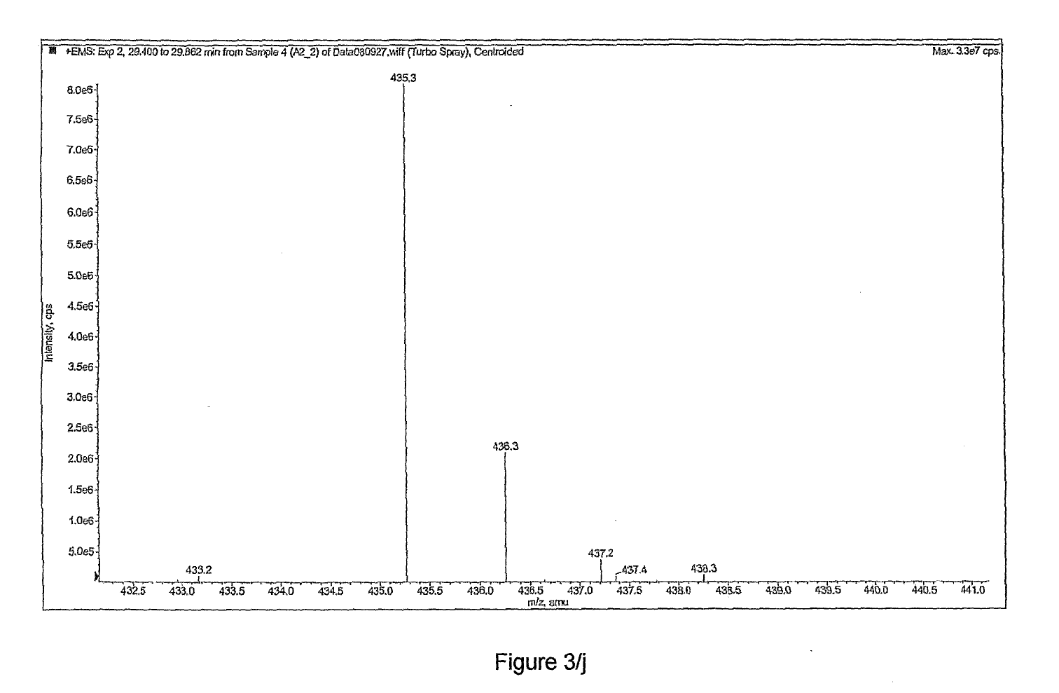

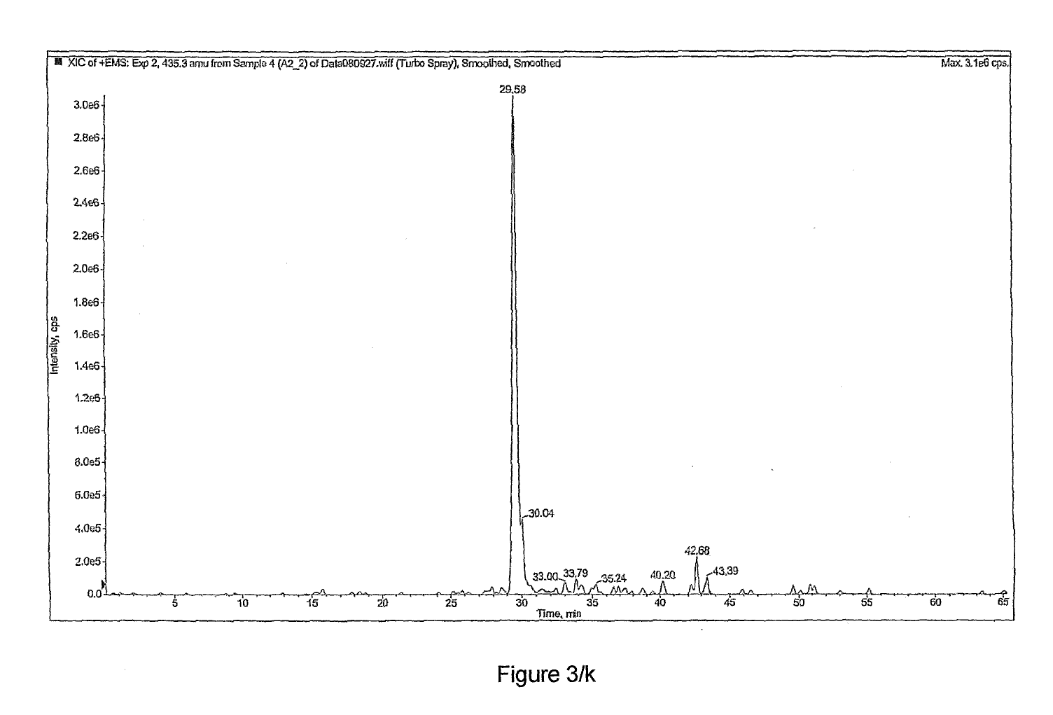

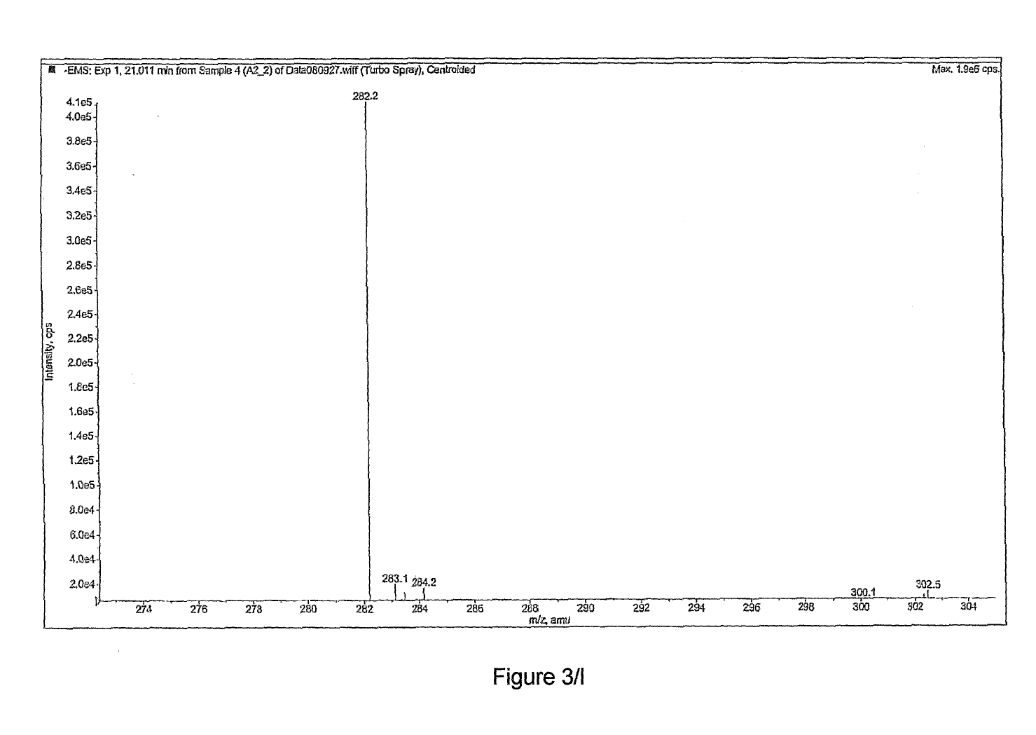

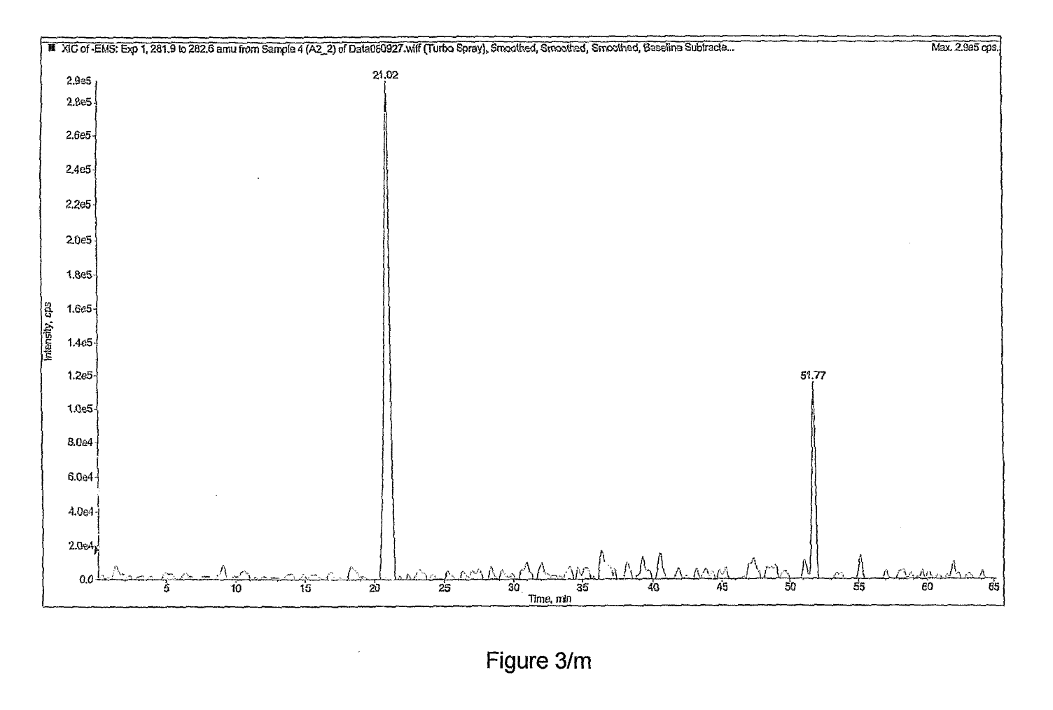

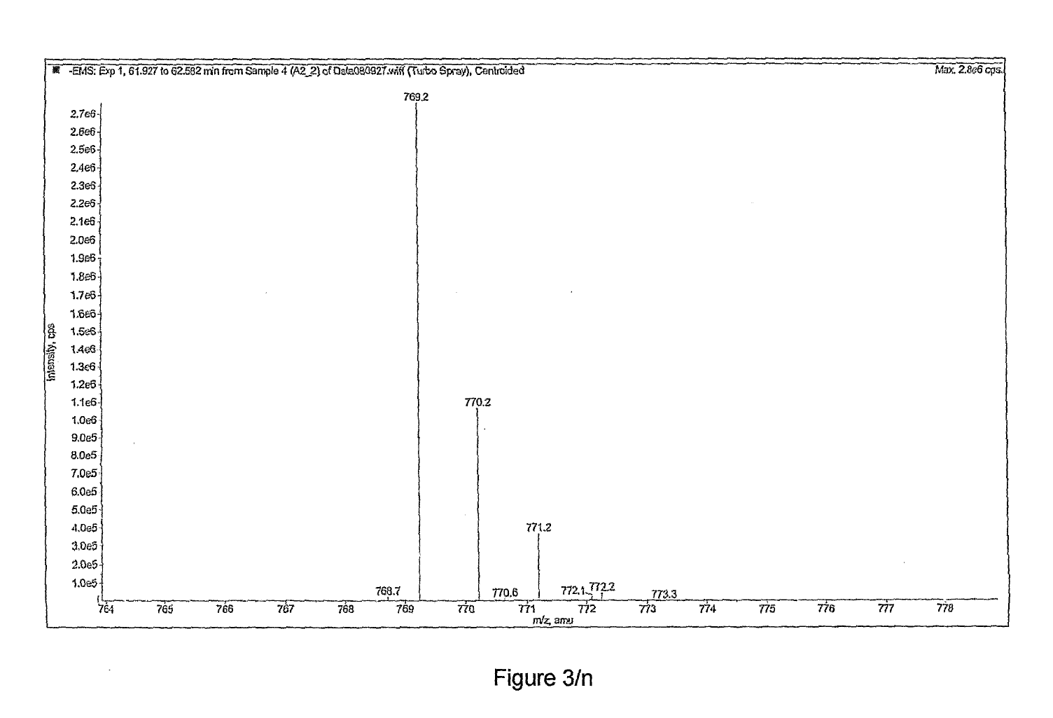

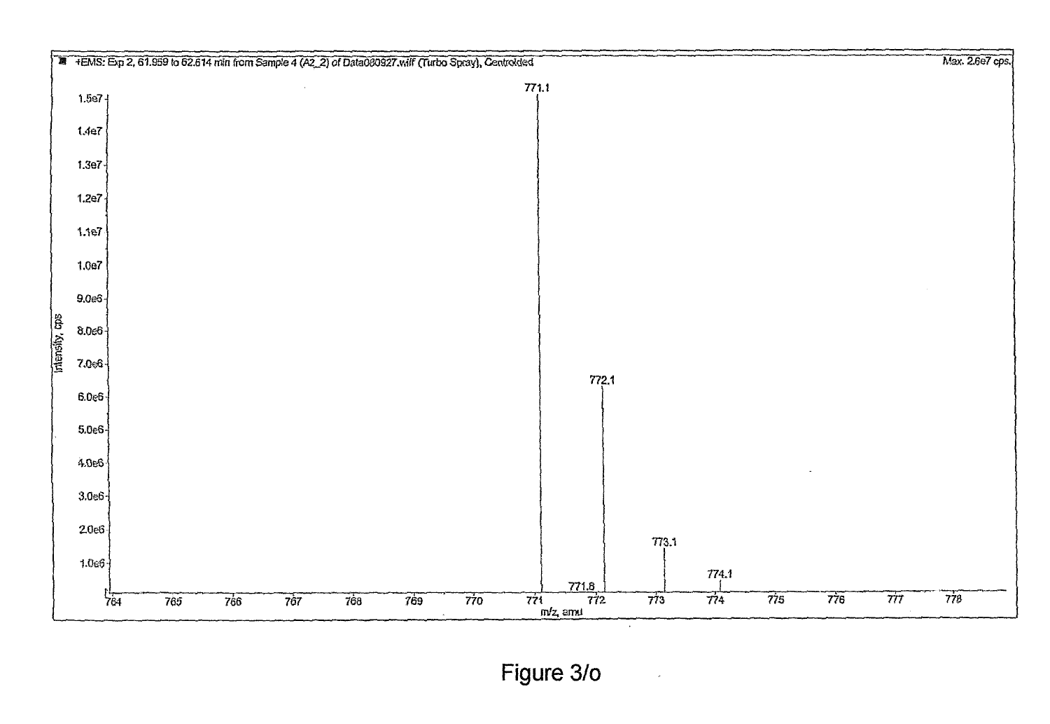

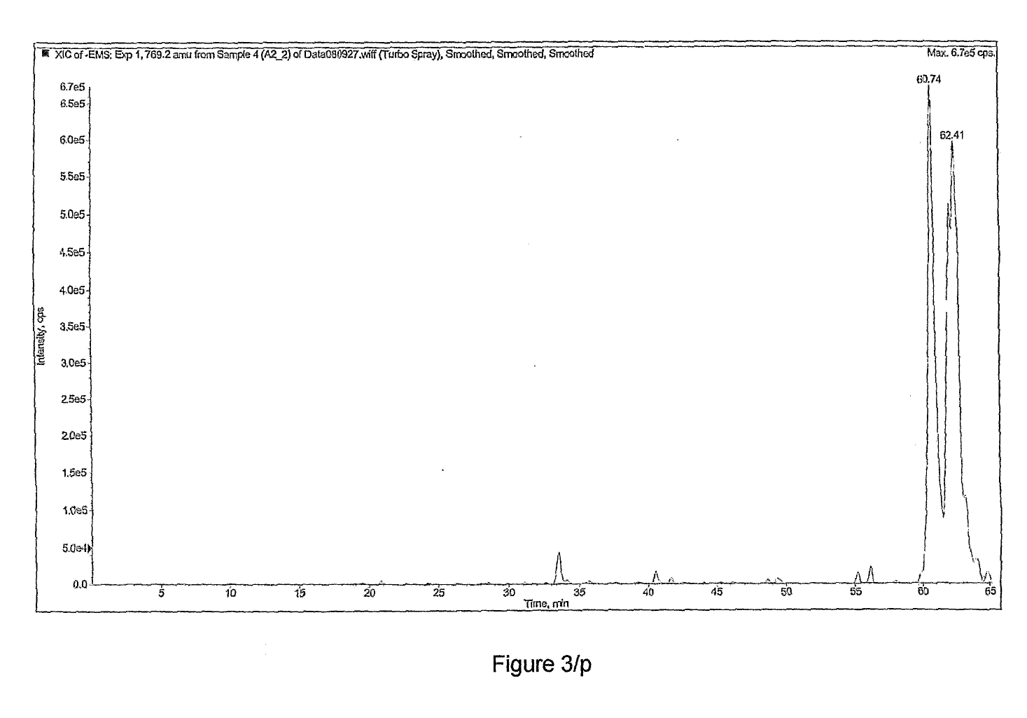

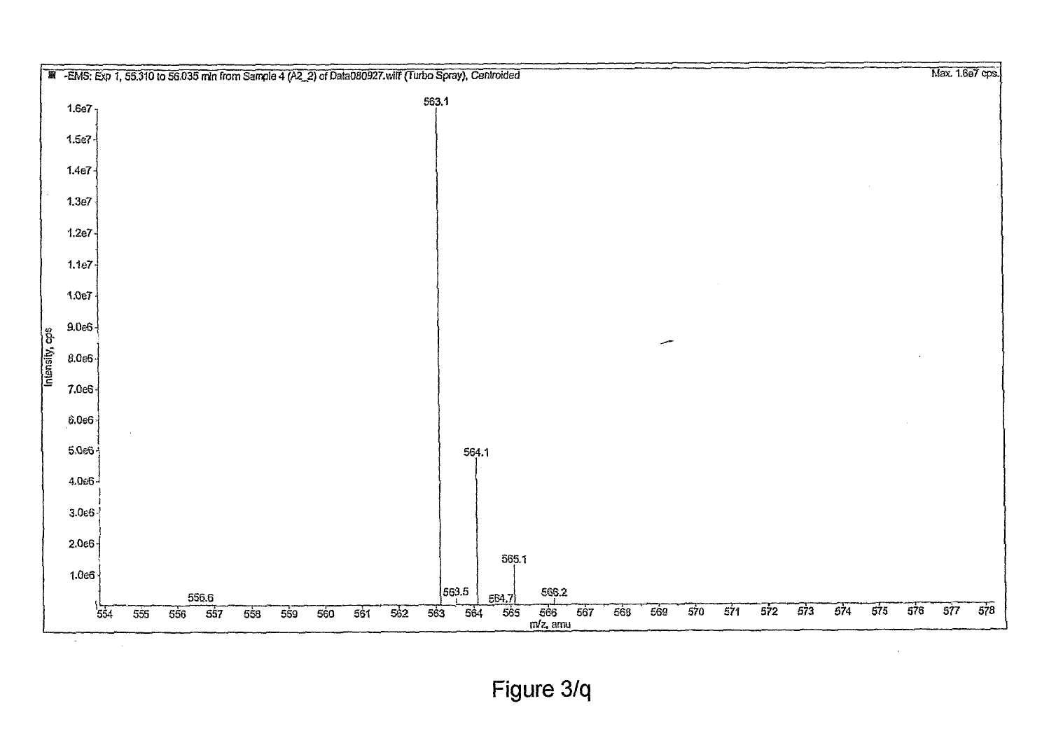

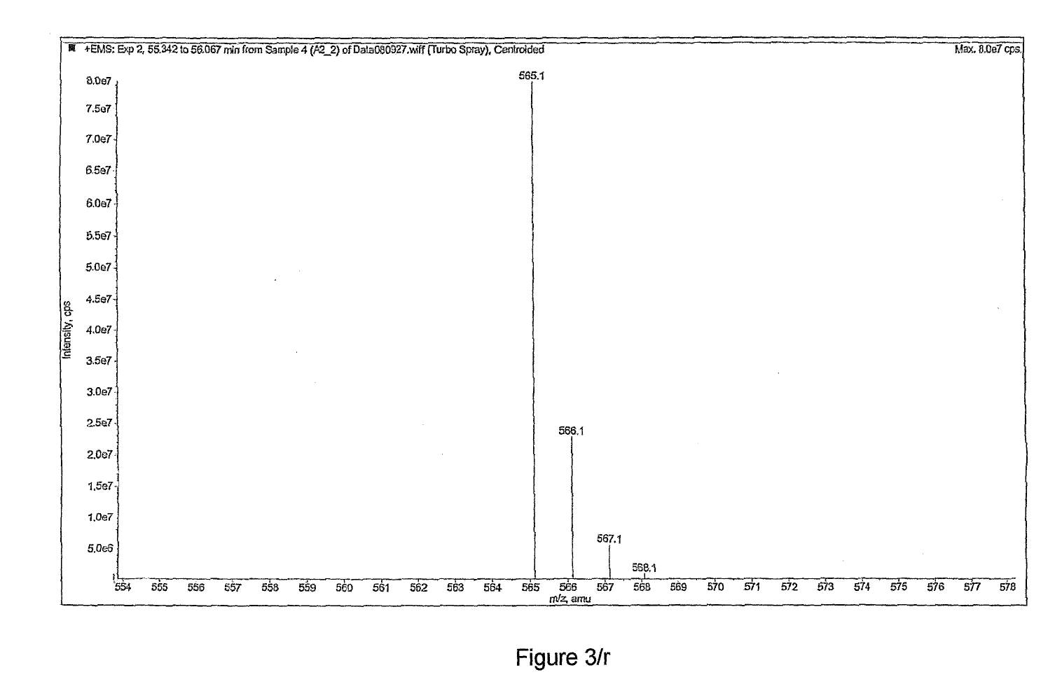

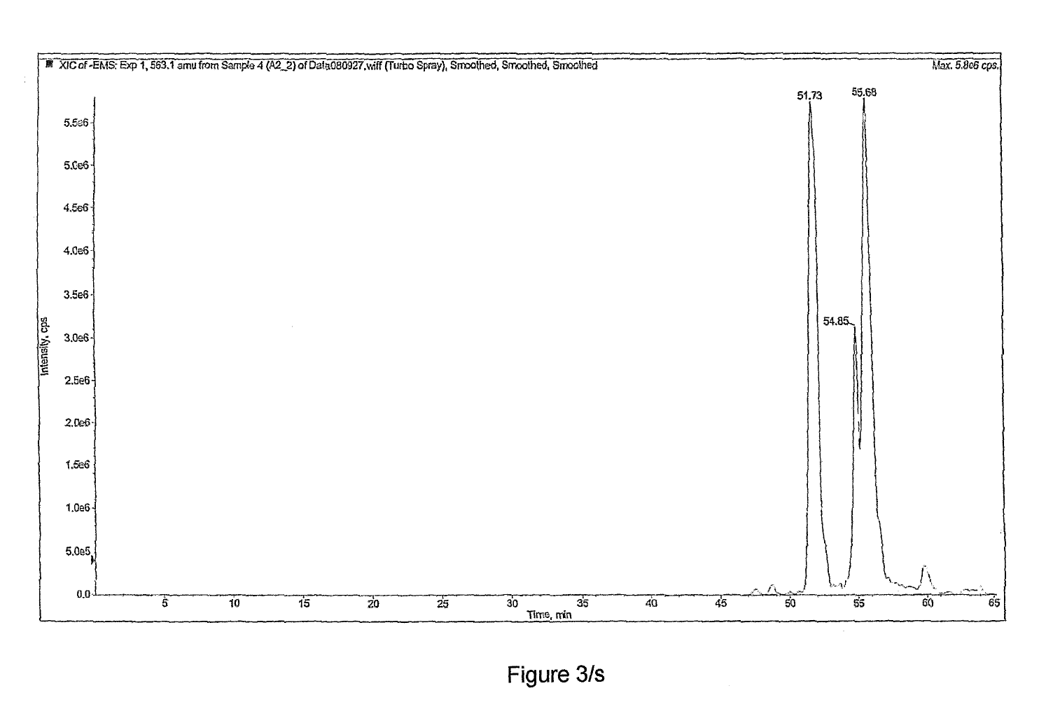

Fraction A2 is a material mixture, FIGS. 3a-3s present masses of some of its components.

The filtrate (fraction A1) was suspended in water, centrifuged, the supernatant decanted, and organic solvent was added. The precipitate was filtered, and dried. The mass of the thus produced fraction E was 15-25% of the starting dry material. In contradiction to the lyophilizate, fraction E is not hygroscopic, and the unpleasant smell disappeared, too.

In the next step fraction E was dissolved in water, was filtered, and

a) if desired, the solution was evaporated to dryness (fraction ES), or
b) the solution was gel-chromatographed, the material, remained in column, was eluted and lyophilized (fraction L).

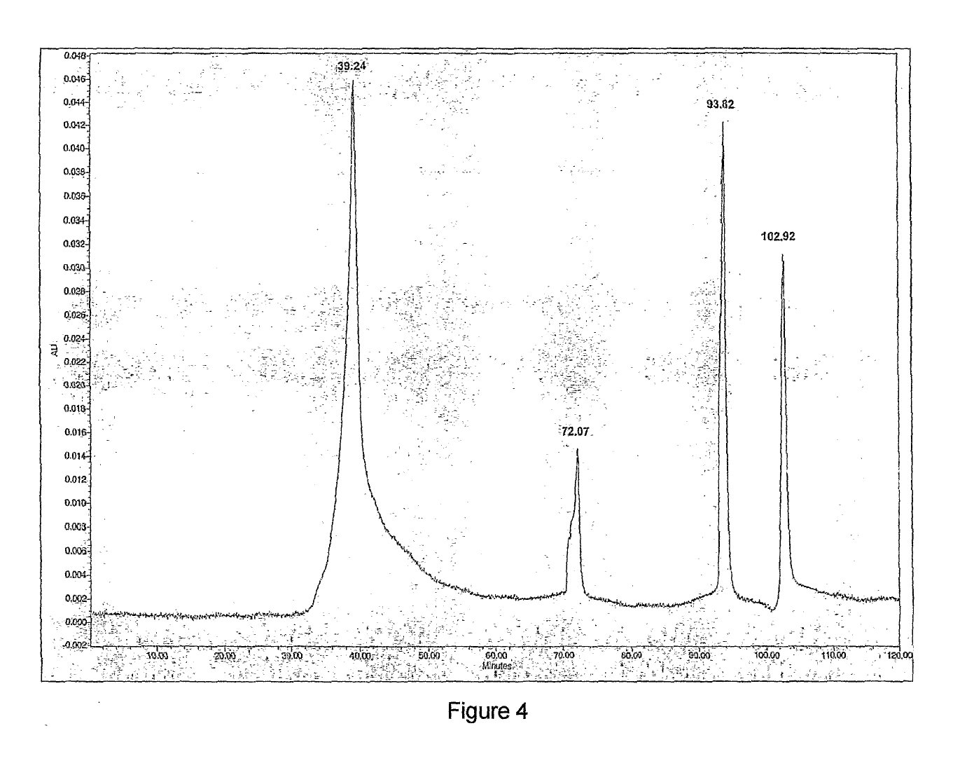

The UV chromatograms of fractions E and ES are nearly identical (FIG. 4).

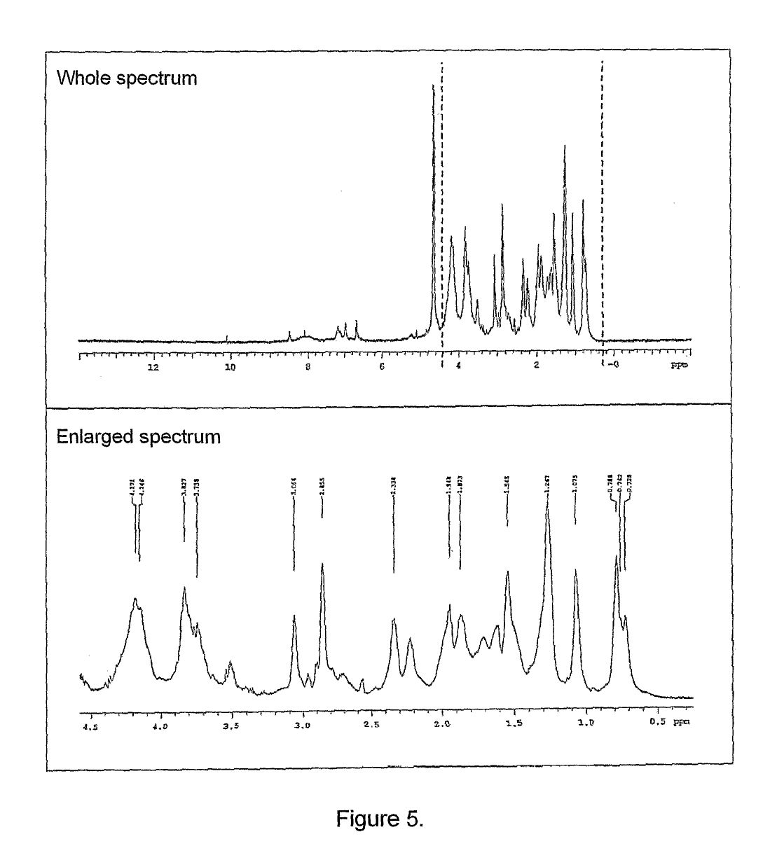

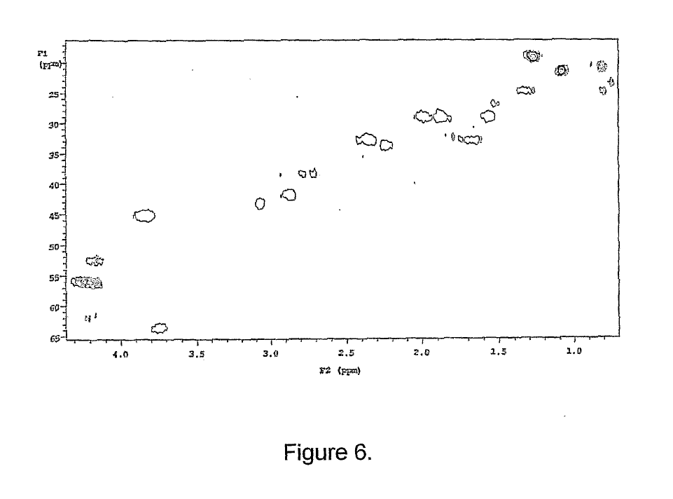

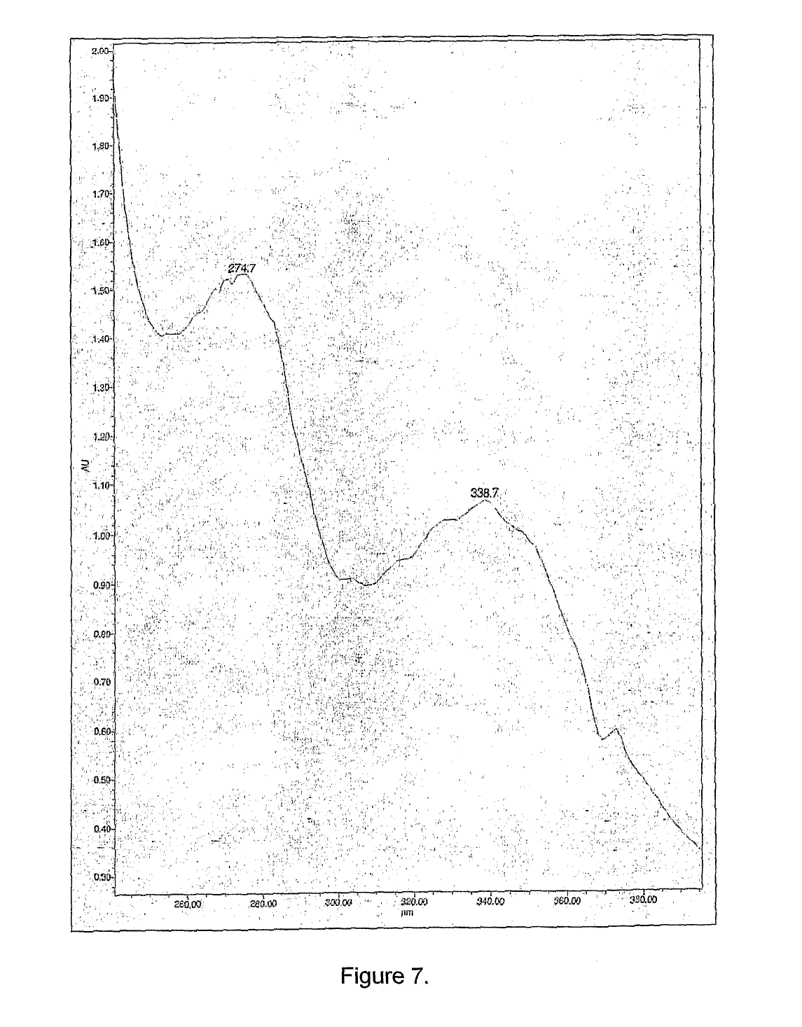

The NMR spectrum of fraction L is shown in FIGS. 5 and 6 (HSQC shot), while the UV spectrum is shown in FIG. 7.

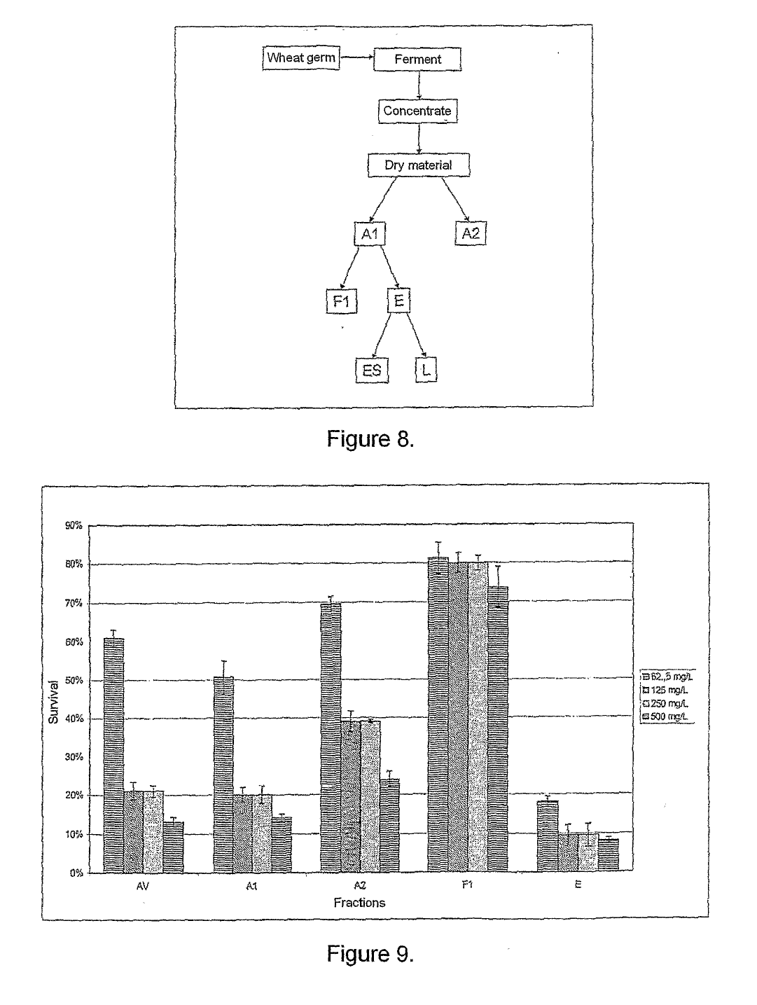

The separation of the fractions is shown schematically in FIG. 8.

The antiproliferative efficacies of the active ingredient (Av) of Avemar®, and the fractions A1, A2, E, ES and L were tested in A431 and SW480 cancer cell lines. The results are shown in FIGS. 9-11.

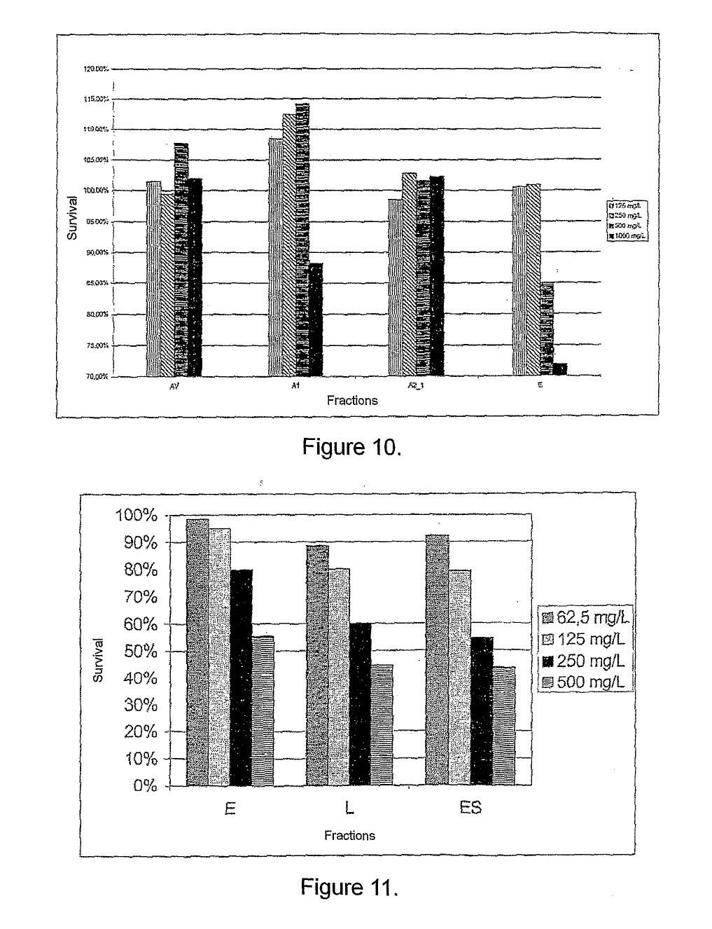

The fraction A1 is more effective than the A2, as shown in FIGS. 9-10.

It can be seen in FIG. 9 that at concentration 500 mg/ml, the fraction E is more effective in A431 cells than the active ingredient of Avemar®.

As seen in FIG. 10, the active ingredient of Avemar® was ineffective in A431 cell line at a concentration of 1000 mg/ml, while when the cells were treated by fraction E at concentrations of 1000 mg/ml and 500 mg/ml, the survivals of the cells were 75% and 85%, respectively.

FIG. 11 shows the effects of the fractions E, ES and L in A431 cells. Fractions ES and L were more effective than fraction E.

We set the target also to explore the mode of action of the ferment and its fractions.

According to the above, the subject of our invention comprises biologically active fractions obtained from wheat germ ferment, particularly the fractions A2, E, ES and L obtained by fractionation of wheat germ ferment, which was obtained by fermenting wheat germ in aqueous medium in the presence of Saccharomyces cerevisiae, and by concentrating and/or dehydrating the fermentation liquid.

The UV chromatogram of fraction A2 basically corresponds to that in FIG. 2.

The mass chromatograms of fraction A2 basically correspond to those in FIGS. 3a-3s.

The UV chromatograms of fractions E and ES basically correspond to that in FIG. 4.

The NMR spectrum of fraction L basically corresponds to that in FIG. 5 and to the HSQC shot in FIG. 6, the UV spectrum basically corresponds to that in FIG. 7.

Fraction A2 can be produced from wheat germ ferment in the way that the wheat germ ferment is dissolved in alcohol, if needed, this alcoholic dissolution can be repeated with the filtrate, and the alcoholic phases are evaporated.

For alcohol, methanol or ethanol, preferably methanol can be used.

To produce fraction E, the wheat germ ferment is dissolved in alcohol, if needed, this alcoholic dissolution can be repeated with the filtrate, the filtrate is suspended in water, then centrifuged, and fraction E is precipitated from the supernatant by an organic solvent. For organic solvent, hexane, ethylacetate, alcohol, while for alcohol, preferably methanol or ethanol, most preferably, methanol can be used.

The fractions ES and L can be produced in the way that fraction E is dissolved in water, filtered, and

a) if desired, the solution is dried (fraction ES), or
b) the solution is gel-chromatographed, the material, remained in the column, is eluted by the use of an appropriate eluent, if needed, the thus resulted solution is neutralized, and, if desired, dried (fraction L).

For gel-filtration chromatography, carbohydrate based gels, preferably agarose-based ones, more preferably, agarose-dextran based gel-filtration materials can be used.

For washing the column, as eluent, diluted acid, preferably, hydrochloric acid, formic acid, acetic acid, apple-vinegar, wine-vinegar, trifluoro-acetic acid, citric acid, tartaric acid, malic acid, ascorbic acid, preferably, 0.1N hydrochloric acid, or bases, preferably, alkali hydroxides, alkaline earth hydroxides-, oxides, ammonium hydroxide can be used.

Provided, the acid or base eluent could not be evaporated, the solution can be neutralized, as it is well-known, by the use of the appropriate acid or base, and the salt precipitated could be removed, as it is also well-known.

The drying of the eluted fraction L can be carried out by vacuum drying, or lyophilisation, preferably, by lyophilisation.

The subject of our invention also comprises pharmaceutical preparations containing, in separate dosage forms, fractions E, or ES, or L and, in certain cases, A2.

Fractions E, ES and L can be formulated preferably in forms of tablets, dragées, granules, sachets, capsules, suspension, emulsion, spray, suppository, ointment, patch, liposome with the application of auxiliary materials and procedures commonly used in pharmaceutical technology.

Fraction A2 can be formulated preferably in forms of capsules, coated tablets, coated dragées, suppository, ointment, patch with the application of auxiliary materials and procedures commonly used in pharmaceutical technology.

The subject of our invention further comprises the use of fractions E and/or ES and/or L, in certain cases, together with fraction A2 for the manufacturing of pharmaceutical preparations with immunomodulatory and antitumor properties, and for production of dietary supplement, medical food or dietary food for special medical purpose for mammals, respectively.

Of the fractions, according to the invention, fractions ES and L can be most preferably used to produce pharmaceutical preparations.

For the treatment and/or prevention of cancer, and/or for modulating pathological immune functions, and/or for preventing the development of infectious diseases by strengthening the immune defense mechanisms, an effective amount of the pharmaceutical preparation containing one or more of the fractions of the invention is administered to the patients.

Of the fractions according to the invention, ES and L have the most advantageous biological activity.

The effective dosage may vary according to the type of disease, the state, age, body weight of the patient. The single daily dosage for a 70 kg weight human being is usually 0.1-10 g A2, and 0.05-10 g E, ES, or L active ingredient, respectively.

The subject of our invention further comprises the application of the biologically active material and its fractions, obtained from wheat germ ferment, on their own, or in combination with other known pharmaceutical preparations for the treatment and/or diagnosis of diseases, in development of which 5′ adenosine monophosphate-activated protein kinase (AMPK) molecules have regulatory role, such as neoplastic diseases (cancer), metabolic syndrome, diabetes, hypertension, obesity, arteriosclerosis, etc.

Further particulars of the invention are described in the examples, without limiting the invention to the examples.

### EXAMPLES

### Example 1

### Isolation of the Fractions

The concentrate (in 25 ml units) of the wheat germ ferment was lyophilized. The weight of the dry material per units was 5.5 g. To this quantity of dry material a total of 100 ml water free methanol (Sigma) was added in several steps. The color of the liquid became brown, while the solid phase became lighter, pale. After ultrasonic shaking, the mixture was filtered by G4-filter, and was washed with methanol.

Further quantity of methanol was added to the filtrate, and the above written step was repeated 4-times until the liquid remained colorless. The methanolic phases were united and evaporated at 60° C. A honey-like material (fraction A2) was obtained. The fraction was kept at −80° C.

The mass of this fraction A2 was approximately 2 g (appr. 37%).

The color of the filtrate (A1) was greyish. After drying with hexane or diisopropyl ether (Sigma) the mass of this fraction was 3 g (54%).

Fraction A2 was investigated by HPLC-UV equipment (PerkinElmer Series 200) connected to a mass detector (AB Sciex Instruments 4000 Q TRAP). Nucleodur Sphinx RP 4.6/150 3 micrometer column (Macherey-Nagel) was used. Eluting conditions:

| Time | Flow-rate | A | B |
|---|---|---|---|
| (min) | microliter/min | (water, 1% HCOOH) | (Methanol, 1% HCOOH) |
| 5.0 | 400.00 | 100.0 | 0.0 |
| 37.0 | 400.00 | 10.0 | 90.0 |
| 40.0 | 400.00 | 10.0 | 90.0 |
| 45.0 | 400.00 | 0.0 | 100.0 |
| 55.0 | 400.00 | 0.0 | 100.0 |
| 65.0 | 400.00 | 100.0 | 0.0 |

To the fraction A1, 90 ml of water was added, and the mixture was suspended by ultrasonic. A part of the solids can not be dissolved either. The suspension was centrifuged (14000 rpm). The supernatant was decanted, and the precipitate (pellet) was suspended in methanol, and filtered, and dried with diisopropyl ether (F1).

Fraction E was precipitated from the supernatant by the addition of 10-times quantity of methanol. The precipitate was filtered with a G4 glass filter. It was dried with diisopropyl ether, and a powder of greyish-white color was obtained. (Fraction E, mass: 1 g (18%)). After evaporation, the filtrate was united with fraction A2.

a) 50 mg portion of fraction E was dissolved in water, and centrifuged (14000 rpm). The supernatant was lyophilized. The mass of the thus obtained fraction was 22.2 mg (fraction ES).
b) 50 mg portion of fraction E was dissolved in water, and centrifuged (14000 rpm). The supernatant was injected onto Superdex 200 10/300 GL column (Sigma) by a HPLC equipment (Waters W2790), and the UV chromatogram was registered (Waters 996 PDA). (Flow-rate: 400 microliter/min, eluent: water).

After isocratic elution, the column was washed with 0.1N HCl (flow-rate: 400 microliter/min). The eluted fraction was lyophilized. The mass of the thus obtained fraction was 8.5 mg (fraction L).

### Example 2

### Consecutive Use of Two Column

Fraction E, obtained as described in Example 1, was injected, under conditions as described, onto Superdex 200 10/300 GL and Superdex 75 10/300 GL columns by the HPLC equipment as described above, with elution parameters as also described above. The columns were washed, as described in Example 1, and the hydrochloric acid solution was lyophilized. The mass of the thus obtained fraction was 9.7 mg (fraction L).

### Example 3

### Modification of the Fractions

1-1 grams of the fractions E, or ES, or L, obtained according to the above written examples, were suspended in formaldehyde solution of 10 mol/l (Sigma). The solutions were dried at room temperature, the dry residues were dissolved in water, and the aqueous solutions were filtered through Sephadex column (Sigma). The excluded phases were collected and dried.

### Example 4

### Cancer Cell Proliferation Assays

The fractions, obtained as described in the Example 1 and 2, were tested in the following cancer cell lines.

A431 human epidermic carcinoma cell line (Giard D J, et al. In vitro cultivation of human tumors: establishment of cell lines derived from a series of solid tumors. J Natl Cancer Inst. 51: 1417-23, 1973.) http://www.lgcstandards-atcc.org/LGCAdvancedCatalogueSearch/ProductDescription/tabid/1068/Default.aspx?AT CCNum=CRL-1555&Template=cellBiology

SW480 Human Colon Carcinoma Cell Line (Leibovitz A, et al. Classification of human colorectal adenocarcinoma cell lines. Cancer Res. 36: 4562-9, 1976.) http://www.lgcstandards-atcc.org/LGCAdvancedCatalogueSearch/ProductDescription/tabid/1068/Defaultaspx?AT CCNum=CCL-228&Template=cellBiology

The cells were inoculated in the medium (ATCC), containing penicillin and streptomycin (Sigma), as described at the web-site: www.atcc.org.

### Methods Used for Testing Antiproliferation Efficacy:

MTT (Buttke T M et al. Use of an aqueous tetrazolium/formazan assay to measure viability and proliferation of lymphokine dependent cell lines. J Immunol Methods 157: 233-8, 1993);

ATP Light Luminescence assay (Gareval H S et al. J Natl cancer Inst. 1986 November; 77(5): 1039-45).

The results of the assays are shown in FIGS. 9-11. FIG. 9 shows the results of the ATP Light Luminescence assay, with 1000 cell/well, after 48 hours of treatment. FIGS. 10-11 show the results obtained by the MTT assay, with 10 000 cell/well, after 48 hours of treatment.

### Example 5

### Kinase Panel Assays

The wheat germ ferment was dissolved in DMSO, and the solution was diluted with water until the DMSO concentration reached 5%. This solution was tested by enzyme assay (kinase panel). The percentage changes of activities as a result of the treatment with the wheat germ ferment are shown in Table 1.

TABLE 1

| % Activity | Target | % Activity | | % Activity | | % Activity | Target |
|---|---|---|---|---|---|---|---|
| Change | ID | Change | Target ID | Change | Target ID | Change | ID |
| −7 | ABL2 | −6 | SRC | −19 | p38α | −12 | JAK2 |
| −6 | AKT1 | −30 | RET | −10 | PKCδ | −10 | MET |
| −84 | AMPK | −10 | BLK | −7 | ZAP70 | −16 | PAK2 |
| −1 | CDK2 | −9 | CDK4 | −14 | SYK | 1 | PAK3 |
| 0 | CDK5 | −7 | COT | −12 | KDR | 1 | PAK4 |
| −10 | CK2α1 | −8 | ERK2 | −7 | AKT2 | 0 | PAK7 |
| −14 | FGFR1 | −18 | FGFR3 | −39 | AURORA | −1 | PGDFRβ |
| | | | | | A | | |
| −12 | LCK | −42 | FLT3 | −11 | AURORA | −5 | RAF1 |
| | | | | | B | | |
| −32 | p70S6K | −7 | GSK3β | −34 | AXL | 3 | RIPK2 |
| −19 | PDK1 | −3 | HCK | −4 | BRAF | −3 | TGFβR1 |
| −2 | PKAcα | 10 | JNK1 | −2 | CDK1 | 7 | TGFβR2 |
| −3 | PKCα | −1 | ASK1 | −9 | c-KIT | 2 | TIE2 |
| −2 | PKCε | −3 | MAPKAPK2 | −5 | DDR2 | −6 | TRKA |
| −24 | PKCμ | −7 | MAPKAPK5 | −17 | FAK | −14 | TRKB |
| −26 | ROCK2 | −7 | MEK1 | 2 | HER2 | 14 | YES1 |
| −45 | RSKI | −3 | MEK2 | 4 | IGF1R | 1 | EGFR |

The ferment highly significantly inhibited (84%) the AMPK target. It also greatly inhibited (34%-45%) the following kinases: p70S6K, RSK1, RET, AURORA A, AXL, and FLT3. It also inhibited (24%, and 26%) the targets, PKC-alpha and ROCK2, respectively.

### Example 6

### Antitumor Effects of Fractions A2 and E, Isolated from Wheat Germ Ferment, in S-180 Murine Sarcoma Tumor Model

In our experiments the comparative antitumor effects of the fraction A2 (denoted as A2) and fraction E (denoted as E), obtained as described in Example 1, and the antitumor effects of the lyophilized wheat germ ferment (denoted as LYO) were investigated in S-180 murine sarcoma tumor model. Antitumor effects of the samples were measured by their effects on tumor growth and on overall survival in S-180 sarcoma bearing mice.

The experiments were done with relatively equal amounts taking into account, i.e. relative to the concentration ratios of the fractions in the wheat germ ferment lyophilizate.

Inbred SPF (specific pathogen free) female BDF1 mice with 22-24 g body weight were used. Animals were given Altromin feed and tap drinking water ad libitum.

S-180 murine tumor was transplanted (Type: sarcoma. Origin: Chester Beatty Cancer Res. Inst., London, UK. Inoculum: tissue. Mode of transplantation: sub cutaneous (s.c.). Host animal: BDF1 (C57B1 female X DBA/2 male) inbred hybride mouse from SPF hygienic quality certified breed).

The transplantation of the tumor was carried out by s.c. transplantation of optimal tumor pieces and/or fragments into the interscapular region by tweezers. Prior to surgery, animals were narcotized by Nembutal (50 mg/kg, i.p.).

Animals were treated orally once daily for 10 days (10×qd). For detecting any toxic effect of the treatments, body weights were systematically registered.

Treatments were started after the appearance of the measurable tumor (7 days after tumor transplantation). After randomization groups of 7-7 animals were formed. Randomization was carried out by measuring each animal's tumor volume thus, getting a mean value for tumor size. Mice, having larger or smaller tumor than that of the mean value, were discarded. The average tumor volumes in the groups were equal.

### Evaluation of Antitumor Effect:

The antitumor effects of the samples were determined by comparing changes of tumor volume and overall survival in the treated and non-treated (control) groups. Digital calipers were used for the continuous measurement of tumour volumes. The determination of tumour volume was done by using the following formula, accepted and used in the literature (Tomayko M. M., Reynolds C. P.: Determination of subcutaneous tumor size in athymic (nude) mice. Cancer Chemother Pharmacol. 24: 148-154, 1989):

V=D²×L×π/6

where V=tumour volume, D=shorter diameter, L=longer diameter.

Animals were observed daily, and measurements of tumor volume was done in every second day.

### Results

### Effects of A2, E and LYO on the Growth of S-180 Sarcoma Tumor:

The results of the experiments show that A2, E, and LYO reduced tumor growth by 48%, 50%, and 53%, respectively.

### Effects of A2, E and LYO on Overall Survival:

A2, E, and LYO lengthened overall survival of sarcoma mice by 46%, 51%, and 43%, respectively.

No toxic effects of the treatments were detected.

1.-36. (canceled)

37. Biologically active fractions of a wheat germ ferment.

38. Biologically active fractions A2, E, ES and L of a wheat germ ferment—where the wheat germ ferment is obtainable by fermenting wheat germ in aqueous medium in the presence of Saccharomyces cerevisiae, and concentrating and/or dehydrating the fermentation liquid—, obtained by fractionation of said wheat germ ferment, where fraction A2 is separated, the residue is fractionated, fraction E is separated and if desired fractionated for obtaining fractions ES and L.

39. Fraction A2 of a wheat germ ferment—where the wheat germ ferment is obtainable by fermenting wheat germ in aqueous medium in the presence of Saccharomyces cerevisiae, and concentrating and/or dehydrating the fermentation liquid—according to claim 37, obtained by that the wheat germ ferment is dissolved in alcohol, filtered, if desired the alcoholic dissolution is repeated several times with the filtrate, and the alcoholic phase is evaporated.

40. Fraction A2, according to claim 39, having an UV chromatogram substantially corresponding to that shown in FIG. 2.

41. Fraction E of a wheat germ ferment—where the wheat germ ferment is obtainable by fermenting wheat germ in aqueous medium in the presence of Saccharomyces cerevisiae, and concentrating and/or dehydrating the fermentation liquid—according to claim 37, obtained by that the wheat germ ferment is dissolved in alcohol, filtered, if desired the alcoholic dissolution is repeated several times with the filtrate, the filtrate is suspended in water then centrifuged, and fraction E is precipitated from the supernatant by organic solvent.

42. Fraction ES of a wheat germ ferment—where the wheat germ ferment is obtainable by fermenting wheat germ in aqueous medium in the presence of Saccharomyces cerevisiae, and concentrating and/or dehydrating the fermentation liquid—according to claim 37, obtained by that fraction E is dissolved in water, filtered, and, if desired the filtrate is dried.

43. Fractions E or ES, according to claim 41, having an UV chromatogram substantially corresponding to that shown in FIG. 4.

44. Fraction L of a wheat germ ferment—where the wheat germ ferment is obtainable by fermenting wheat germ in aqueous medium in the presence of Saccharomyces cerevisiae, and concentrating and/or dehydrating the fermentation liquid—according to claim 37, obtained by that the fraction E is dissolved in water, if desired filtered, the thus obtained solution is chromatographed by gel-filtration, the material remained in the column is washed out by an appropriate eluent, if desired the thus obtained solution is neutralized and, if desired dried.

45. Fraction L, according to claim 44, having an NMR spectrum substantially corresponding to that shown in FIG. 5, an HSQC shot shown in FIG. 6, and an UV spectrum substantially corresponding to that shown in FIG. 7.

46. Process for the preparation of fraction A2 as defined in claim 39 from a wheat germ ferment—where the wheat germ ferment is obtainable by fermenting wheat germ in aqueous medium in the presence of Saccharomyces cerevisiae, and concentrating and/or dehydrating the fermentation liquid—, characterized by that the wheat germ ferment is dissolved in alcohol, filtered, if desired the alcoholic dissolution is repeated several times with the filtrate, and the alcoholic phase is evaporated.

47. Process for the preparation of fraction E as defined in claim 41 from a wheat germ ferment—where the wheat germ ferment is obtainable by fermenting wheat germ in aqueous medium in the presence of Saccharomyces cerevisiae, and concentrating and/or dehydrating the fermentation liquid—, characterized by that the wheat germ ferment is dissolved in alcohol, filtered, if desired the alcoholic dissolution is repeated several times with the filtrate, the filtrate is suspended in water then centrifuged, and fraction E is precipitated from the supernatant by organic solvent.

48. Process for the preparation of fraction ES as defined in claim 42 from a wheat germ ferment—where the wheat germ ferment is obtainable by fermenting wheat germ in aqueous medium in the presence of Saccharomyces cerevisiae, and concentrating and/or dehydrating the fermentation liquid—, characterized by that fraction E is dissolved in water, filtered, and, if desired the filtrate is dried.

49. Process for the preparation of fraction L as defined in claim 44 from a wheat germ ferment—where the wheat germ ferment is obtainable by fermenting wheat germ in aqueous medium in the presence of Saccharomyces cerevisiae, and concentrating and/or dehydrating the fermentation liquid—, characterized by that the fraction E is dissolved in water, if desired filtered, the thus obtained solution is chromatographed by gel-filtration, the material remained in the column is washed out by an appropriate eluent, if desired the thus obtained solution is neutralized and, if desired dried.

50. The process according to claim 47, characterized by that as organic solvents hexane, ethyl acetate or alcohols are used.

51. The process according to claim 46, characterized by that as alcohol methanol or ethanol, preferably, methanol is used.

52. The process according to claim 49, characterized by that as gel-filtration chromatography, carbohydrate based gels, preferably agarose-based ones, more preferably, agarose-dextran based gel-filtration materials are used.

53. The process according to claim 49, characterized by that as eluent diluted acids or bases, preferably, hydrochloric acid, formic acid, acetic acid, apple-vinegar, wine-vinegar, trifluoro-acetic acid, citric acid, tartaric acid, malic acid, ascorbic acid, or alkali hydroxides, alkaline earth hydroxides-, oxides, ammonium hydroxide, preferably 0.1N hydrochloride is used.

54. The process according to claim 49, characterized by that the drying is carried out by vacuum-drying, preferably by lyophilization.

55. Pharmaceutical preparation containing as active ingredient one or more fractions as defined in claim 37.

56. Pharmaceutical preparation according to claim 55, containing as active ingredient fraction E, or ES, or L or A2.

57. Pharmaceutical preparation according to claim 55, containing as active ingredient fraction E, or ES, or L and fraction A2 in separate dosage forms.

58. Preparation according to claim 55, characterized by that the fractions E, ES, L are formulated in forms of tablets, dragées, granules, sachets, capsules, suspension, emulsion, spray, suppository, ointment, patch, liposome.

59. Preparation according to claim 55, characterized by that the fraction A2 is formulated in forms of capsules, coated tablets, coated dragées, suppository, ointment, patch.

60. Use of fraction E, or ES, or L as defined in claim 37, if desired together with fraction A2, for the production of pharmaceutical preparations having immunostimulatory, immunomodulatory and antitumor properties.

61. Use of fraction E, or ES, or L as defined in claim 37, if desired together with fraction A2, for the production of dietary supplement, medical food or dietary food for special medical purpose for mammals, respectively.

62. Use of fraction ES as defined in claim 37, if desired together with fraction A2, for the production of pharmaceutical preparations having immunostimulatory, immunomodulatory and antitumor properties.

63. Method of treatment and/or prevention of cancer, characterized by administering to the patient an effective amount of the pharmaceutical preparation or pharmaceutical preparations containing one or more of the fractions as defined in claim 37.

64. Treatment according to claim 63, characterized by administering to the patient an effective amount of the pharmaceutical preparation containing fraction E, or ES, or L.

65. Treatment according to claim 63, characterized by administering to the patient an effective amount of a combination of a pharmaceutical preparation containing as active ingredient fraction E, or ES, or L, and the pharmaceutical preparation containing as active ingredient fraction A2.

66. Treatment according to claim 63, characterized by administering to the patient an effective amount of the pharmaceutical preparation containing fraction ES.

67. Process for the stimulation of immune functions or for the modulation of pathological immune functions, characterized by administering to the patient an effective amount of the pharmaceutical preparation or pharmaceutical preparations, containing one or more of the fractions as defined in claim 37.

68. Process according to claim 67, characterized by administering to the patient an effective amount of the pharmaceutical preparation containing fraction E, or ES, or L.

69. Process according to claim 67, characterized by administering to the patient an effective amount of the combination of a pharmaceutical preparation containing as active ingredient fraction E, or ES, or L, and a pharmaceutical preparation containing as active ingredient fraction A2.

70. Process according to claim 67, characterized by administering to the patient an effective amount of the pharmaceutical preparation containing fraction ES.

71. Use of a biologically active material and its fractions, obtained from wheat germ ferment, on their own, or in combination with other known pharmaceutical preparations for the treatment of diseases, in development of which 5′ adenosine monophosphate-activated protein kinase (AMPK) molecules have a role.

72. Use of the wheat germ ferment, obtained by fermenting wheat germ in aqueous medium in the presence of Saccharomyces cerevisiae, and by concentrating and/or dehydrating the fermentation liquid, and the biologically active fractions obtained by the fractionation of said ferment on their own, or in combination with other known pharmaceutical preparations for the treatment of diseases, in development of which 5′ adenosine monophosphate-activated protein kinase (AMPK) molecules have a role.
